(19) United States
(12) Patent Application Publication
(10) Pub. No.: US 2025/0354903 A1
(43) Pub. Date: Nov. 20, 2025

(54) APPARATUS, SYSTEMS AND METHODS FOR CORNEA RECOVERY

(71) Applicant: University of Iowa Research Foundation, Iowa City, IA (US)

(72) Inventors: Christopher Sales, Coralville, IA (US); Geb Thomas, Iowa City, IA (US); Kara Ann Johnson, Iowa City, IA (US)

(21) Appl. No.: 18/872,159

(22) PCT Filed: Jun. 8, 2023
(86) PCT No.: PCT/US2023/024858

§ 371 Date: Dec. 5, 2024

Related U.S. Application Data
(60) Provisional application No. 63/350,192, filed on Jun. 8, 2022.

(51) Int. Cl. G01N 1/28

(52) U.S. Cl. CPC G01N 1/286; G01N 2001/2873

(57) ABSTRACT

The disclosed apparatus, systems and methods relate to cadaver cornea removal. The device for cornea recovery comprising a barrel, at least one curved needle extending from a distal end of the barrel, a collar, at least one straight needle extending from a distal end of the collar, a guard comprising openings for insertion of the at least one curved needles and the at least one straight needle, wherein the collar is configured for slidable communication with the barrel and wherein the barrel is configured for rotational communication with the guard.

### CROSS-REFERENCE TO RELATED APPLICATION(S)

This application claims priority to U.S. Provisional Application No. 63/350,192 filed Jun. 8, 2022 and entitled “Apparatus, Systems and Methods for Cornea Recovery,” which is hereby incorporated by reference in its entirety under 35 U.S.C. § 119 (e).

### TECHNICAL FIELD

The disclosure relates to devices, systems and methods for removal of corneal tissue.

### BACKGROUND

The disclosure relates to apparatus, systems and methods for cornea recovery. The current recovery technique of manually excising the cornea from its cadaveric donor by a trained eye bank technician with a pair of scissors and forceps. In the current procedure, the eye-a hollow, pressurized organ—is punctured with a scalpel through the sclera (the white of the eye), and a circumferential cut is made. The main challenge arises when cutting the tissue to free it from the iris after the scleral wall has been punctured and the eyeball has lost its shape and rigidity. As the cut reaches completion, it becomes increasingly difficult to manipulate the tissue without distorting the cornea's natural shape. Excessive distortion of the cornea permanently damages the endothelial cell layer, whose health after the recovery is a surrogate for procedural success or failure. Most recover technicians have surgical dexterity and surgical judgment that are much less developed than experienced corneal surgeons.

Thus, there is a need in the art for a device to improve the speed and accuracy of cornea removal.

### BRIEF SUMMARY

Discussed herein are various devices, systems and methods relating to cornea removal. According to certain embodiments, the disclosed devices, systems and methods replace the current recovery technique of manually excising the cornea from its cadaveric donor by a trained eye bank technician with a pair of scissors and forceps. In #3443973 the current procedure, the eye—a hollow, pressurized organ—is punctured with a scalpel through the sclera (the white of the eye), and a circumferential cut is made. The main challenge arises when cutting the tissue to free it from the iris after the scleral wall has been punctured and the eyeball has lost its shape and rigidity. As the cut reaches completion, it becomes increasingly difficult to manipulate the tissue without distorting the cornea's natural shape. Excessive distortion of the cornea permanently damages the endothelial cell layer, whose health after the recovery is a surrogate for procedural success or failure.

Most recover technicians have surgical dexterity and surgical judgment that are much less developed than experienced corneal surgeons. The device facilitates corneal tissue recovery by automating critical elements of the current process. It reduces the potential for corneal tissue damage during excision by statically fixating the scleral wall. Additionally, it may also facilitate excision of the corneoscleral rim and iris together to enable ex vivo tissue dissection under a laboratory microscope, avoiding the current practice of in-the-field in situ tissue dissection without the benefit of magnification. Lastly, it will make the corneoscleral rim dimensions and shape consistent, which can be critical for downstream tissue processing for endothelial keratoplasty with a microkeratome.

The device will increase the rate of success of corneal excision, providing important economic benefits. Of the 85,000 corneas recovered annually in the US, 30% are not suitable for transplant. One-third to half of the unsuitable recoveries are lost due to damage caused during the recovery procedure. Medicare, Medicaid, and private insurances in the US reimburse between $3500 and $5500 for each corneal tissue transplanted. Thus, conservatively, 8,500 corneas worth $29.75 to $46.75 million in revenue are lost by US eye banks annually.

In Example 1, a scleral fixation recovery device comprising: an elongate central shaft; a sliding collar defining a lumen, the lumen shaped to accommodate the elongate central shaft; and a guard comprising a guard lumen sized for the dissection of scleral tissue, wherein the guard is configured to be secured to scleral tissue for dissection.

Example 2 relates to the device of Example 1, further comprising at least one first needle fixedly attached at a distal end of the elongate central shaft and at least one second needle extending from a distal end of the sliding collar.

Example 3 relates to the device of any of Examples 1-2, wherein the at least one first needle is a curved needle and the at least one second needle is a straight needle.

Example 4 relates to the device of any of Examples 1-3, wherein the guard comprises one or more first openings configured to accept the at least one first needle and one or more second openings configured to accept the at least one second needle, and wherein the one or more first openings allow for rotational movement of the at least one first needle to engage scleral tissue and wherein the at least one second needle locks the device in place.

Example 5 relates to the device of any of Examples 1-4, further comprising a corneal support mechanism comprising a concave surface in fluidic communication with a vacuum configured to apply suction to a cornea surface.

Example 6 relates to the device of any of Examples 1-5, wherein the elongate central shaft comprises one or more longitudinal grooves the sliding collar comprises one or more longitudinal tongues, and wherein the sliding collar is rotationally secured about the elongate central shaft via tongue and groove interactions.

Example 7 relates to the device of any of Examples 1-6, wherein the elongate central shaft comprises one or more needle housings shaped to hold the at least one first needle.

In Example 8, a device for cornea recovery comprising: a barrel; at least one curved needle extending from a distal end of the barrel; a collar; at least one straight needle extending from a distal end of the collar; and a guard comprising openings for insertion of the at least one curved needles and the at least one straight needle, wherein the collar is configured for slidable communication with the barrel and wherein the barrel is configured for rotational communication with the guard.

Example 9 relates to the device of Example 8, wherein the guard is conical and comprises one or more first openings to accommodate the at least one curved needle and one or more second openings to accommodate the at least one straight needle.

Example 10 relates to the device of any of Examples 8-9, further comprising a corneal support mechanism comprising a concave surface in fluidic communication with a vacuum.

Example 11 relates the device of any of Examples 8-10, wherein the barrel comprises at least one groove along an exterior surface of the barrel and wherein the collar comprises at least one tongue along an interior surface of the collar, and wherein the at least one groove and at least one tongue interface for rotationally fixed slidable communication of the collar along the barrel.

Example 12 relates to the device of any of Examples 8-11, wherein the at least one straight needle is affixed within an opening defined in a distal end of the at least one tongue.

Example 13 relates to the device of any of Examples 8-12, wherein the at least one straight needle is disposed radially about the distal end of the collar.

Examples 14 relates to the device of any of Examples 8-13, wherein the at least one curved needle is disposed radially about the distal end of the barrel.

In Example 15, a cornea removal device comprising: a barrel defining a first lumen; one or more curved needles disposed radially about and extending from a distal end of the barrel; at least one groove along an exterior surface of the barrel; a collar defining a second lumen, the second lumen configured to accept the barrel for slidable communication; one or more straight needles disposed radially about and extending from a distal end of the collar; at least one tongue along the interior surface of the collar, wherein the at least one groove and at least one tongue interface for rotationally fixed slidable communication of the collar along the barrel.

Example 16 relates to the device of Example 15, further comprising a corneal support mechanism comprising a concave surface in fluidic communication with a vacuum.

Example 17 relates to the device of any of Examples 15-16, further comprising a distal guard comprising one or more first openings configured to accept the one or more curved needles and one or more second openings configured to accept the one or more straight needles, and wherein the one or more first openings allow for rotational movement of the one or more curved needles to engage eye tissue.

Example 18 relates to the device of any of Examples 15-17, wherein the barrel comprises one or more needle housings shaped to affix the one or more curved needles to the barrel.

Example 19 relates to the device of any of Examples 15-18, wherein each of the one or more straight needles are disposed within an opening defined in a distal surface of the at least one tongue.

Example 20 relates to the device of any of Examples 15-19, further comprising a lighting element operationally integrated into the barrel.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A is a side view of a scleral fixation recovery device prior to urging the collar distally, according to one implementation.

FIG. 1B is a side view of the scleral fixation recovery device of FIG. 1A with the collar locked in the distal position.

FIG. 1C is a side view of the scleral fixation recovery device of FIGS. 1A-1B showing the cutting of the cornea with a blade.

FIG. 1D is a side view of the scleral fixation recovery device of FIGS. 1A-1C following the removal of the cornea.

FIG. 2 is an exploded view of a scleral fixation recovery device, according to one implementation.

FIG. 3A is a side view of the barrel, according to one implementation.

FIG. 3B is a perspective of the barrel, according to one implementation.

FIG. 3C is a top view of the barrel, according to one implementation.

FIG. 4 is a perspective view of the collar, according to one implementation.

FIG. 5 is a perspective view of the distal end of the scleral fixation recovery device, according to one implementation.

### DETAILED DESCRIPTION

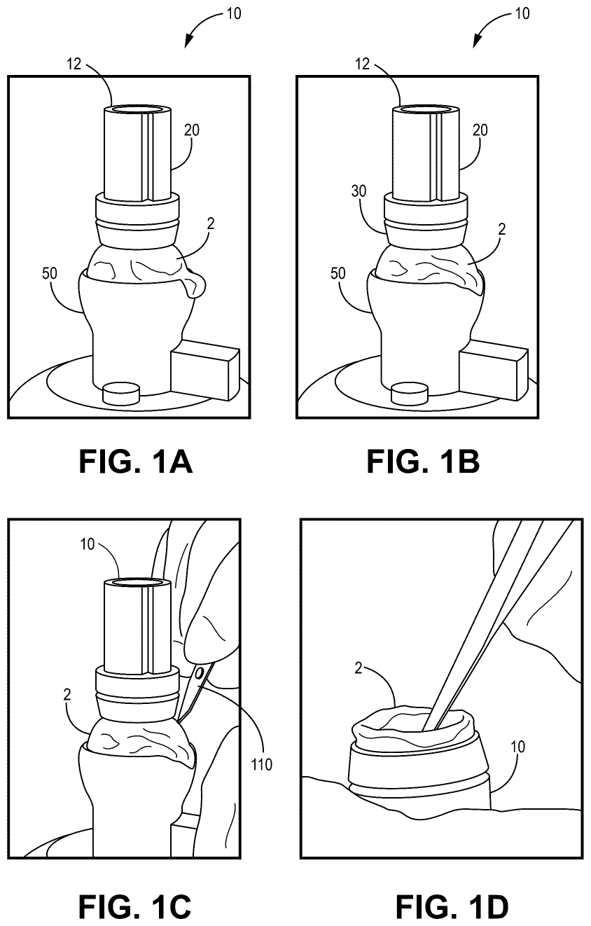

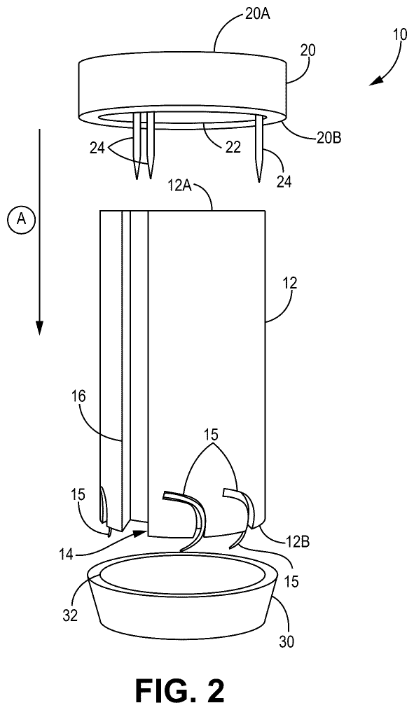

The disclosed technology relates to a device and associated systems and methods configured to mechanical secure corneal tissue at the front of the eye and provides a guide to cut the tissue. Various implementations can be used to secure scleral tissue at the front of the eye for corneal recovery in situ or in an enucleated eye in the lab, as would be readily apparent. One implementation of the scleral fixation recovery device 10 applied to an eye 2 is depicted in FIGS. 1A-1D. As shown in FIGS. 1A-1D and in exploded view in FIG. 2, in various implementations the device 10 comprises an elongate central shaft 12 or barrel 12 having a proximal end 12A, a distal end 12B and an elongate lumen 14 disposed therethrough. In these implementations, one or more curved or crescent-shaped needles 15, such as suture needles are fixedly attached at the distal end 12B so as to extend beyond the distal end 12B for use in the procedure. While the present discussion includes implementations featuring needles, further implementations can contemplate the use of other tools or methods of securing and/or cutting the underlying or subject tissue, such as pins, blades and other securing and cutting devices understood in the art such as a trephine. Various alternative implementations may include a metallic ring with curved needle-like teeth in place of needles 15.

Continuing with FIG. 2, in these implementations, a substantially circular sliding collar 20 also with a barrel lumen 22 is provided, wherein the barrel lumen 22 is sized to accommodate the barrel 12. These implementations of the collar 20 further comprise one or more straight needles 24 disposed radially about the distal side 20B of the collar 20.

FIG. 2 also depicts a suture needle retention ring 30 or guard 30 having an internal lumen 32 with a diameter 32′ sized to encircle the cornea 2 for the performance of the desired procedure, as would be understood. The a distal guard 30 is optionally conical and comprising an internal lumen 32 configured to accommodate the disclosed components, including the curved and straight needles for use in the described procedure.

In certain implementations, and as shown in FIG. 1A-1C, a corneal support mechanism 50 comprising of concave surface 52 in fluidic communication with a vacuum (not shown) is configured to support the eye 2 and apply gentle suction to the epithelial surface of cornea to further prevent tissue distortion during the entirety of the corneal recovery process.

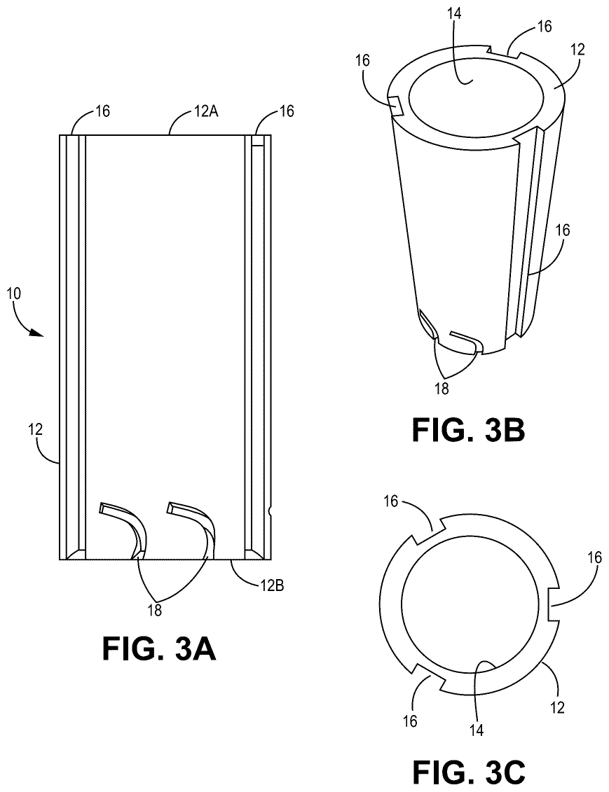

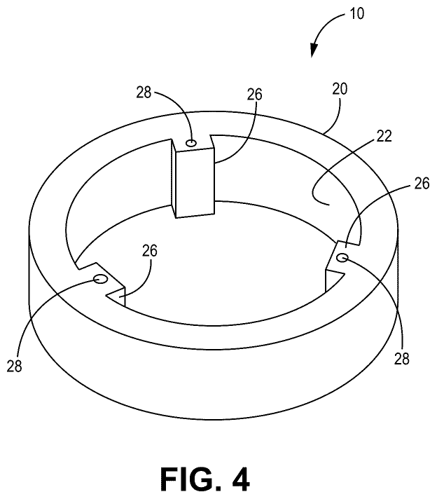

Returning to FIG. 2 and as also shown in FIGS. 3A-3C, the barrel 12 according to certain implementations comprises one or more longitudinal grooves 16 on the exterior surface of the barrel 12. As shown in FIG. 4, the collar 20 according to certain implementations comprises one or more corresponding longitudinal tongues 26 on the interior of the collar 20, such that when the collar 20 is placed on the barrel 12, the device 10 is configured to rotationally secure the collar 20 to the barrel 12 via one or more tongue-and-groove interactions between the grooves 16 and tongues 26, as would be readily understood. That is, according to these implementations, the collar 20 is in rotationally-fixed slidable communication with the barrel 12 such that it can be urged proximally or distally.

As shown in FIGS. 3A-B, in certain implementations the barrel 12 further comprises one or more needle housings 18 configured to accommodate and affix the curved needles 15.

FIG. 4 depicts a view of the collar 20 according to certain implementations, wherein the tongues 26 having openings 28 disposed therein for the fixed attachment of the needles 24, such as via glue, as would be readily appreciated.

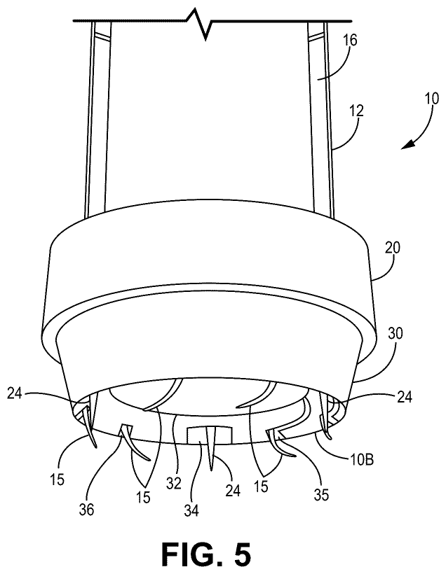

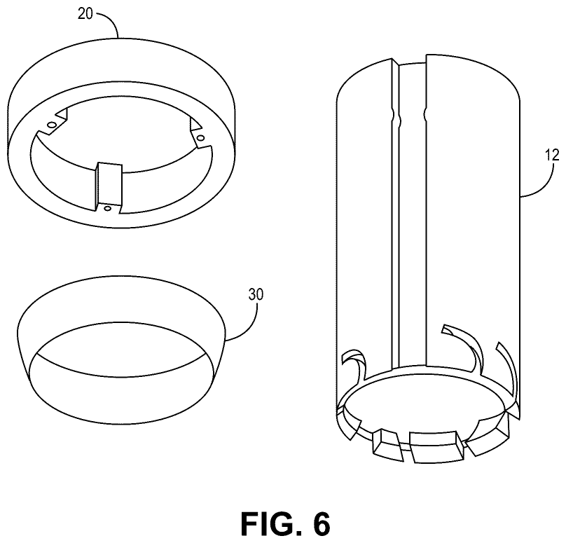

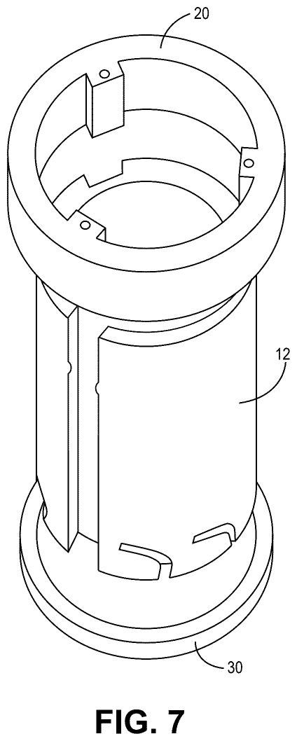

Returning to FIGS. 1A-1D, in use, as the device 10 is brought in contact with the eye 2, the guard 30 is centered about the cornea and then the barrel 12 is rotated to engage the crescent-shaped needles 15 via guard openings 35 (shown in FIG. 5), the needles 15 being oriented in the direction of rotational motion into the eye tissue. The engagement of the curved needles 15 clamp the eye tissue against the distal end of the guard to fixate the scleral tissue, protecting the cornea from deformation during the cutting process. In certain implementations, the needles 15 may be controlled by a lever or other similar device such that when the device 10 is in contact with the cornea the needles 15 rotate out of the distal end of the barrel 12, through the cornea tissue, and around until the tips of the needles 15 are in contact with the distal end of the barrel 12, thereby pinning the cornea tissue to the device 10.

In various alternative implementations, the needles 15 are replaced with a wire that becomes bent as to projects out of the barrel 12 in such a manner that the wire curls through the cornea tissue until the wire is pressed against the bottom of the barrel 12 and the tissue is secured therewith.

Subsequently, as shown in FIG. 1B, the collar 20 is advanced distally such that the straight needles 24 emerge from openings 34 in the guard 30 at the distal device end 10B, as shown in FIG. 5. That is, after the curved needles 15 have secured the scleral tissue, the collar 20 may be lowered to engage the scleral tissue via the straight needles 24. This prevents counter-rotation of the scleral fixator, allowing the operator to release the rotational force required to hold the device 10 in place.

In these implementations, a cut is then performed with either a trephine or a series of scalpels 110, as is shown in FIG. 1C. In the case of the trephine, after the device 10 is locked into place, the trephine is inserted into the lumen 14 of the barrel 12 or around the device's guard 30, and rotated while exerting a light downward force to slice each layer of tissue. Alternatively, a trephine that slides over the outer device 10 may be used.

In the case of scalpels, one or multiple scalpels 110 can be used to puncture the scleral wall around the outside of the device 10. These remain in the tissue while another scalpel is used to complete the cut or the scalpel(s) rotate while being withdrawn from the eye 2. Finally, the device 10 is lifted away from the eye 2 with the corneal tissue attached, thereby completing the corneal recovery, as is shown in FIG. 1D.

In certain implementations, a concave-shaped suction device is placed on the cornea 2, as is shown in FIGS. 1A-1C at 50. The operator then holds the device 10 in place while lowering the locking mechanism into the locked position. The operator then uses either a trephine or a series of scalpels as described above to complete the corneal excision.

In various additional implementations, a lighting element is provided or otherwise operationally integrated into the device 10.

The device is used by first assembling the scleral fixator and rotational lock together. The rotational lock is designed so it can be fixed onto the scleral fixator in only the correct orientation. After assembly, the device is placed onto the eye, around the cornea. The operator twists the device clockwise, which engages the top layers of tissue without puncturing the eye and pulls the eye towards the device.

In certain implementations, the various components of the device 10 other than the needles are constructed of plastic, and the needles 15, 24 are affixed thereto with glue. In further implementations, elements of the device 10 may change to reduce the cost of production and facilitate sterilization. The device 10 may be cast, cut or extruded from metal or other material. The needles 15, 24 may be incorporated into the device 10 components or fixated in another manner. The number of needles 15, 24 in the device 10 components may be changed.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Although the disclosure has been described with reference to preferred embodiments, persons skilled in the art will recognize that changes may be made in form and detail without departing from the spirit and scope of the disclosed apparatus, systems and methods.

What is claimed is:

1. A scleral fixation recovery device comprising:
(a) an elongate central shaft;
(b) a sliding collar defining a lumen, the lumen shaped to accommodate the elongate central shaft; and
(c) a guard comprising a guard lumen sized for the dissection of scleral tissue,
wherein the guard is configured to be secured to scleral tissue for dissection.

2. The device of claim 1, further comprising at least one first needle fixedly attached at a distal end of the elongate central shaft and at least one second needle extending from a distal end of the sliding collar.

3. The device of claim 2, wherein the at least one first needle is a curved needle and the at least one second needle is a straight needle.

4. The device of claim 3, wherein the guard comprises one or more first openings configured to accept the at least one first needle and one or more second openings configured to accept the at least one second needle, and wherein the one or more first openings allow for rotational movement of the at least one first needle to engage scleral tissue and wherein the at least one second needle locks the device in place.

5. The device of claim 1, further comprising a corneal support mechanism comprising a concave surface in fluidic communication with a vacuum configured to apply suction to a cornea surface.

6. The device of claim 1, wherein the elongate central shaft comprises one or more longitudinal grooves the sliding collar comprises one or more longitudinal tongues, and wherein the collar is rotationally secured about the elongate central shaft via tongue and groove interactions.

7. The device of claim 1, wherein the elongate central shaft comprises one or more needle housings shaped to hold the at least one first needle.

8. A device for cornea recovery comprising:
(a) a barrel;
(b) at least one curved needle extending from a distal end of the barrel;
(c) a collar;
(d) at least one straight needle extending from a distal end of the collar; and
(e) a guard comprising openings for insertion of the at least one curved needles and the at least one straight needle,
wherein the collar is configured for slidable communication with the barrel and wherein the barrel is configured for rotational communication with the guard.

9. The device of claim 8, wherein the guard is conical and comprises one or more first openings to accommodate the at least one curved needle and one or more second openings to accommodate the at least one straight needle.

10. The device of claim 9, further comprising a corneal support mechanism comprising a concave surface in fluidic communication with a vacuum.

11. The device of claim 8, wherein the barrel comprises at least one groove along an exterior surface of the barrel and wherein the collar comprises at least one tongue along an interior surface of the collar, and wherein the at least one groove and at least one tongue interface for rotationally fixed slidable communication of the collar along the barrel.

12. The device of claim 11, wherein the at least one straight needle is affixed within an opening defined in a distal end of the at least one tongue.

13. The device of claim 8, wherein the at least one straight needle is disposed radially about the distal end of the collar.

14. The device of claim 8, wherein the at least one curved needle is disposed radially about the distal end of the barrel.

15. A cornea removal device comprising:
(a) a barrel defining a first lumen;
(b) one or more curved needles disposed radially about and extending from a distal end of the barrel;
(c) at least one groove along an exterior surface of the barrel;
(d) a collar defining a second lumen, the second lumen configured to accept the barrel for slidable communication;
(e) one or more straight needles disposed radially about and extending from a distal end of the collar; and
(f) at least one tongue along the interior surface of the collar,
wherein the at least one groove and at least one tongue interface for rotationally fixed slidable communication of the collar along the barrel.

16. The device of claim 15, further comprising a corneal support mechanism comprising a concave surface in fluidic communication with a vacuum.

17. The device of claim 15, further comprising a distal guard comprising one or more first openings configured to accept the one or more curved needles and one or more second openings configured to accept the one or more straight needles, and wherein the one or more first openings allow for rotational movement of the one or more curved needles to engage eye tissue.

18. The device of claim 15, wherein the barrel comprises one or more needle housings shaped to affix the one or more curved needles to the barrel.

19. The device of claim 15, wherein each of the one or more straight needles are disposed within an opening defined in a distal surface of the at least one tongue.

20. The device of claim 15, further comprising a lighting element operationally integrated into the barrel.
